(19) United States
(12) Patent Application Publication
(10) Pub. No.: US 2026/0324995 A1
(43) Pub. Date: Oct. 22, 2026

(54) PULSE WAVE ESTIMATION DEVICE, PULSE WAVE ESTIMATION METHOD, STATE ESTIMATION SYSTEM, AND STATE ESTIMATION METHOD

(71) Applicant: Mitsubishi Electric Corporation, Tokyo (JP)

(72) Inventors: Yudai NAKAMURA, Tokyo (JP); Ryohei MURACHI, Tokyo (JP); Atsushi MATSUMOTO, Tokyo (JP); Shintaro WATANABE, Tokyo (JP)

(73) Assignee: Mitsubishi Electric Corporation, Tokyo (JP)

(21) Appl. No.: 19/115,560

(22) PCT Filed: Nov. 29, 2022
(86) PCT No.: PCT/JP2022/043835

§ 371 Date: Mar. 26, 2025

(51) Int. Cl. A61B 5/024; A61B 5/00; G01J 5/00

(52) U.S. Cl. CPC A61B 5/02433; A61B 5/7278; A61B 5/7485; G01J 5/0025; A61B 2576/00; G01J 2005/0077

(57) ABSTRACT

A pulse wave estimation device includes processing circuitry configured to: acquire an image of a person photographed; detect a skin area from the image; set a plurality of measurement areas in the skin area; specify a first point and a second point in the image; generate first to fourth luminance signals; generate a first pulse wave source signal by combining the first luminance signal and the second luminance signal; and generate a second pulse wave source signal by combining the third luminance signal and the fourth luminance signal; generate a pulse wave signal indicating the pulse wave of the person by subtracting one of the first pulse wave source signal and the second pulse wave source signal from the other; and estimate the pulse wave of the person on a basis of the pulse wave signal.

### TECHNICAL FIELD

The present disclosure relates to a pulse wave estimation device, a pulse wave estimation method, a state estimation system, and a state estimation method.

### BACKGROUND ART

The pulse wave detection device described in Patent Literature 1 subtracts, for example, in an image of a user photographed in a visible light band by an RGB camera, luminance of a red component that has a small pulse wave component of the user and varies depending on movement of the head of the user from luminance of a green component that has a large pulse wave component of the user and varies depending on movement of the head of the user. The pulse wave detection device removes an error in estimating the pulse wave of the user caused by the movement of the head of the user, namely, noise by the subtraction, thereby improving the accuracy of estimating the pulse wave.

### CITATION LIST

### Patent Literature

Patent Literature 1: WO 2016/006027 A

### SUMMARY OF INVENTION

### Technical Problem

However, in the above-described pulse wave detection device, for example, in order to monitor the pulse rate of a user sleeping at night, it is necessary to use a near-infrared camera capable of photographing in the near-infrared wavelength band instead of the above-described RGB camera capable of photographing in a visible light band. The pulse wave detection device has a disadvantage that the use of the near infrared camera does not allow the pulse wave to be estimated by subtraction between the luminance of the green component and the luminance of the red component.

An object of the present disclosure is to provide a pulse wave estimation device, a pulse wave estimation method, a state estimation system, and a state estimation method capable of estimating a pulse wave of a subject even while using an image captured under near-infrared light.

### Solution to Problem

In order to solve the above disadvantage, a pulse wave estimation device according to the present disclosure includes: an image acquisition unit to acquire an image in which a person is photographed under near-infrared light; a skin area detecting unit to detect a skin area from the image, the skin area being a position of skin of the person; a measurement area setting unit to set a plurality of measurement areas in the skin area, the plurality of measurement areas being areas for measuring luminance of the skin of the person; a point specifying unit to specify a first point and a second point in one of the plurality of measurement areas in a direction in which a pulse wave of the person is propagated in the image; a luminance signal generating unit to, for a first distribution and a second distribution having mutually different correspondence relationships between a position on coordinates and a magnitude of weighting specified by the position on the coordinates, generate a first luminance signal indicating luminance weighted by the first distribution at the first point and a second luminance signal indicating luminance weighted by the first distribution at the second point by superimposing the luminance of the first point and the luminance of the second point on the first distribution and to generate a third luminance signal indicating luminance weighted by the second distribution at the first point and a fourth luminance signal indicating luminance weighted by the second distribution at the second point by superimposing the luminance of the first point and the luminance of the second point on the second distribution; a pulse wave source signal generating unit to generate a first pulse wave source signal by combining the first luminance signal and the second luminance signal and to generate a second pulse wave source signal by combining the third luminance signal and the fourth luminance signal; a pulse wave signal generating unit to generate a pulse wave signal indicating a pulse wave of the person by subtracting one of the first pulse wave source signal and the second pulse wave source signal from the other; and a pulse wave estimating unit to estimate the pulse wave of the person on the basis of the pulse wave signal.

### Advantageous Effects of Invention

According to a pulse wave estimation device of the present disclosure, it is possible to improve accuracy of estimating a pulse wave of a subject even while using an image captured under near-infrared light.

### BRIEF DESCRIPTION OF DRAWINGS

FIG. 1 is a functional block diagram of a pulse wave estimation device MSD according to a first embodiment.

FIG. 2A is a diagram illustrating an image Im according to the first embodiment. FIG. 2B is a diagram illustrating a measurement area R of the first embodiment. FIG. 2C is a diagram illustrating a skin area S of the first embodiment.

FIG. 3 is a diagram illustrating basic operations up to generation of a first luminance signal KI1 to a fourth luminance signal KI4 of the first embodiment.

FIG. 4 is a diagram illustrating basic operations up to a pulse wave signal MY of the first embodiment.

FIG. 5 is a diagram illustrating detailed operations up to generation of a first pulse wave source signal MM1 to a second pulse wave source signal MM2 of the first embodiment.

FIG. 6 is a diagram illustrating detailed operations up to generation of the pulse wave signal MY of the first embodiment.

FIG. 7 is a graph illustrating the structures of a weight function A and a weight function B of the first embodiment.

FIG. 8 is a graph illustrating luminance signals of a comparative example.

FIG. 9 is a diagram illustrating a hardware configuration of the pulse wave estimation device MSD of the first embodiment.

FIG. 10 is a diagram illustrating a hardware configuration based on implementation by software of the pulse wave estimation device MSD of the first embodiment.

FIG. 11 is a flowchart illustrating the operation of the pulse wave estimation device MSD of the first embodiment.

FIG. 12 is a diagram illustrating a configuration of a pulse wave estimation device MSD according to a modification of the first embodiment.

FIG. 13 is a functional block diagram of a pulse wave estimation device MSD according to a second embodiment.

FIG. 14 is a flowchart illustrating the operation of the pulse wave estimation device MSD of the second embodiment.

FIG. 15 is a diagram illustrating a first luminance signal KI1 (R1) to a fourth luminance signal KI4 (R1) of the pulse wave estimation device MSD of the second embodiment and operations up to generation of the first luminance signal KI1 (R1) to the fourth luminance signal KI4 (R2).

FIG. 16 is a diagram illustrating operations of the pulse wave estimation device MSD of the second embodiment up to generation of an average pulse wave signal HMY.

FIG. 17 is a functional block diagram of a pulse wave source signal generating unit MMS of a third embodiment.

FIG. 18 is a flowchart illustrating the operation of the pulse wave estimation device MSD of the third embodiment.

FIG. 19 is a functional block diagram of a measurement area setting unit KRS, a point specifying unit TTO, and a luminance signal generating unit KSS of a fourth embodiment.

FIG. 20 is a diagram illustrating a measurement area R, a first point P1 to a fourth point P4, and a first luminance signal KI1 to a fourth luminance signal KI4 according to the fourth embodiment.

FIG. 21 is a flowchart illustrating the operation of the pulse wave estimation device MSD of the fourth embodiment.

FIG. 22 is a functional block diagram of a state estimation system JSS of a fifth embodiment.

### DESCRIPTION OF EMBODIMENTS

Embodiments of a pulse wave estimation device according to the present disclosure will be described.

### First Embodiment

### First Embodiment

A pulse wave estimation device MSD according to a first embodiment will be described.

Hereinafter, in order to facilitate description and understanding, a plurality of names is collectively referred to by one reference sign. For example, a plurality of names of “measurement area R1”, “measurement area R2”, “measurement area R3”, “measurement area R4”, . . . may be collectively referred to with one reference sign “R”.

### Function of First Embodiment

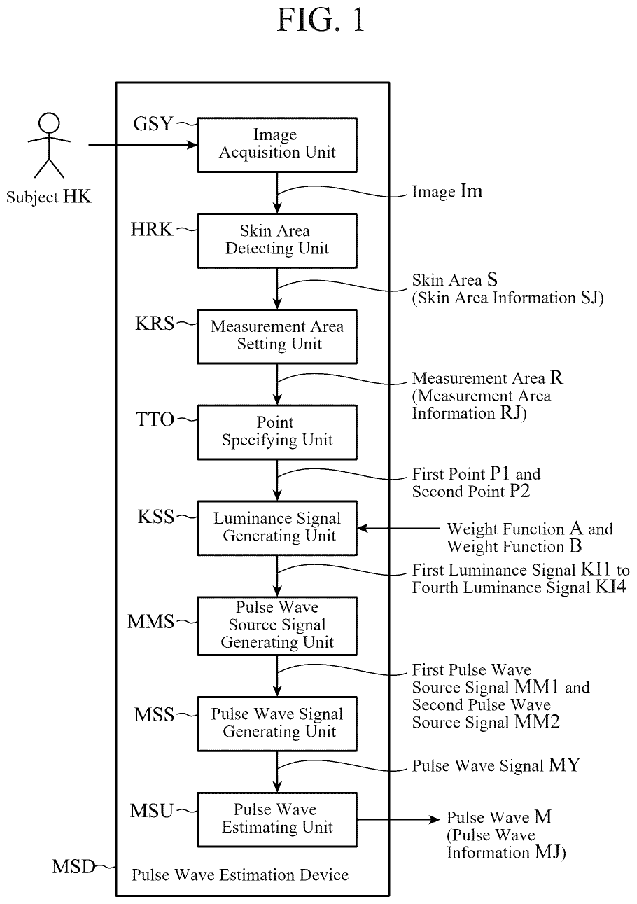

FIG. 1 is a functional block diagram of the pulse wave estimation device MSD according to the first embodiment.

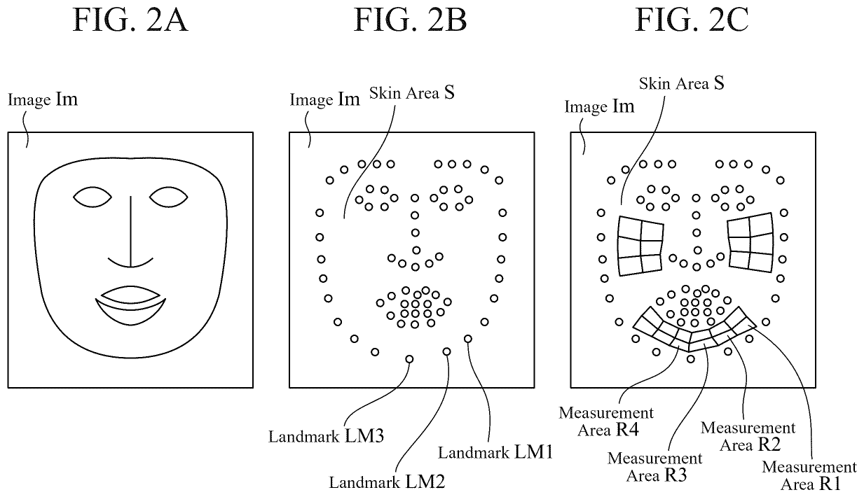

FIG. 2 is a diagram illustrating images Im, measurement areas R, and skin areas S of the first embodiment.

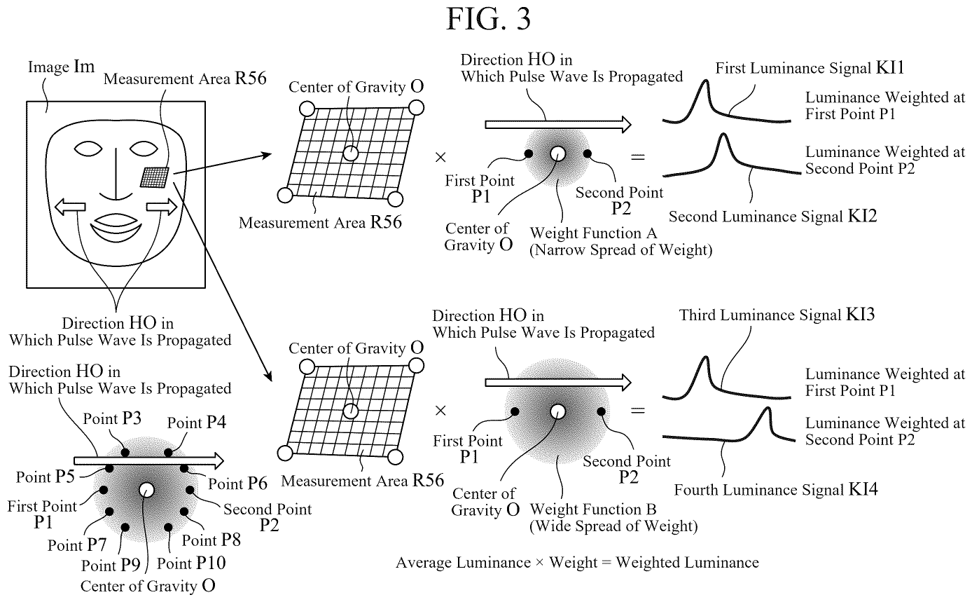

FIG. 3 is a diagram illustrating basic operations up to generation of a first luminance signal KI1 to a fourth luminance signal KI4 of the first embodiment.

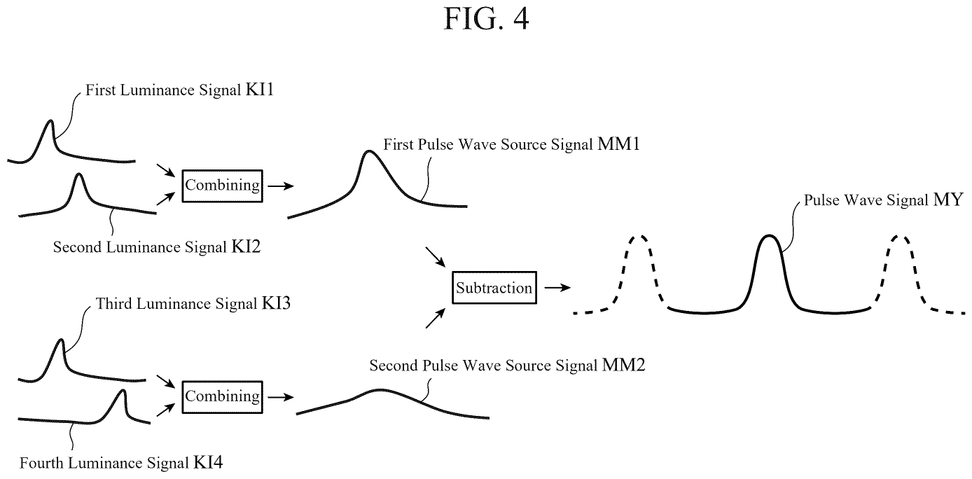

FIG. 4 is a diagram illustrating basic operations up to a pulse wave signal MY of the first embodiment.

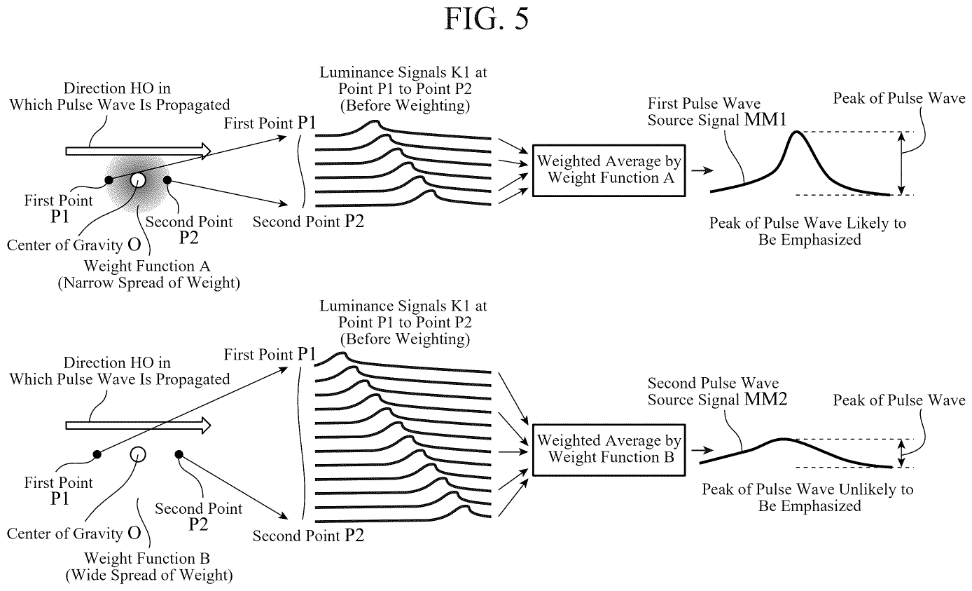

FIG. 5 is a diagram illustrating detailed operations up to generation of a first pulse wave source signal MM1 to a second pulse wave source signal MM2 of the first embodiment.

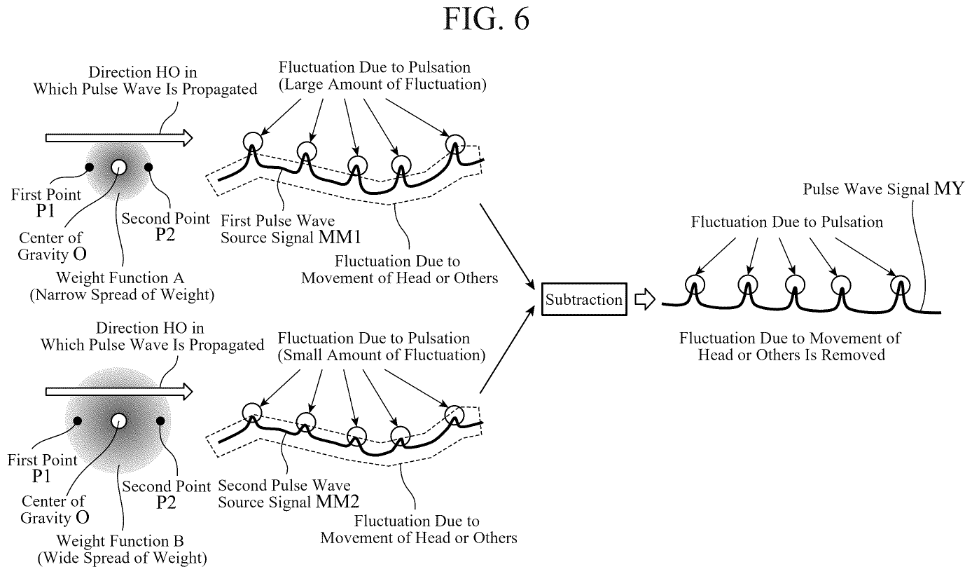

FIG. 6 is a diagram illustrating detailed operations up to generation of the pulse wave signal MY of the first embodiment.

Hereinafter, the function of the pulse wave estimation device MSD of the first embodiment will be described by referring to FIGS. 1 to 6.

As illustrated in FIG. 1, the pulse wave estimation device MSD of the first embodiment includes an image acquisition unit GSY, a skin area detecting unit HRK, a measurement area setting unit KRS, a point specifying unit TTO, a luminance signal generating unit KSS, a pulse wave source signal generating unit MMS, a pulse wave signal generating unit MSS, and a pulse wave estimating unit MSU.

The image acquisition unit GSY corresponds to the “image acquisition unit”, the skin area detecting unit HRK corresponds to the “skin area detecting unit”, the measurement area setting unit KRS corresponds to the “measurement area setting unit”, the point specifying unit TTO corresponds to the “point specifying unit TTO”, the luminance signal generating unit KSS corresponds to the “luminance signal generating unit”, the pulse wave source signal generating unit MMS corresponds to the “pulse wave source signal generating unit”, the pulse wave signal generating unit MSS corresponds to the “pulse wave signal generating unit”, and the pulse wave estimating unit MSU corresponds to the “pulse wave estimating unit”.

The image acquisition unit GSY acquires an image Im (also illustrated in FIG. 2) in which a subject HK who is at least one person is photographed under the near-infrared light (for example, using a near-infrared light camera). Since the image Im is captured under near-infrared light, the image Im is a single-color image (for example, a monochrome image or an image of only one color out of a plurality of colors).

Incidentally, the term “near-infrared light” does not refer only to near-infrared light in a narrow sense but also refers to a single color (for example, green light) of visible light in a broad sense, for example.

The skin area detecting unit HRK detects a skin area S (also illustrated in FIG. 2B) that is the position of the skin of the subject HK, for example, the area of the skin of the face of the subject HK in the image Im by setting landmarks LM1, LM2, LM3, . . . in the image Im by a known method in the related art. The skin area detecting unit HRK generates skin area information SJ indicating a skin area S of the subject HK.

Specifically, the skin area information SJ indicates, for example, whether or not the skin area S of the subject HK is included in the image Im as well as the position and the size of the skin area S of the subject HK in the image Im.

The measurement area setting unit KRS sets, in the skin area S of the subject HK, a plurality of measurement areas R1, R2, R3, R4, . . . (also illustrated in FIG. 2C) that are areas for measuring the luminance of the skin of the subject HK so as to be used for estimation of a pulse wave M of the subject HK. The measurement area setting unit KRS generates measurement area information RJ indicating the measurement areas R.

The plurality of measurement areas R1, R2, R3, R4, . . . are, for example, quadrilateral (illustrated in FIG. 2C).

Specifically, the measurement area information RJ indicates, for example, the position and the size of each of the measurement areas R in the image Im and indicates, for example, coordinate positions of four vertexes of the measurement area R that is a quadrilateral.

As illustrated in FIG. 3, the point specifying unit TTO specifies a first point P1 and a second point P2 in the measurement area R (for example, in a measurement area R56) in a direction HO in which the pulse wave M of the subject HK is propagated in the image Im.

Incidentally, “to specify the first point P1 and the second point P2 in the direction HO in which the pulse wave M of the subject HK is propagated in the image Im” should be interpreted in a broad sense and includes at least the following:
(1) Specifying at least one of the first point P1, a point P3, a point P5, a point P7, or a point P9 and one of the second point P2, a point P4, a point P6, a point P8, or a point P10 (specifying two or more points), as illustrated in the lower left of FIG. 3, for example, (1A) in the direction HO in which the pulse wave M of the subject HK is propagated in the image Im or (1B) in the direction HO in which the pulse wave M of the subject HK will propagate in the image Im; and
(2) In order to specify the above (1), to designate an area (for example, an area where the first point P1, the second point P2, the point P5, and the point P6 may be included) in which two or more points of the first point P1, the second point P2, and the points P3 to P10 may be included.

Note that, as suggested in the lower left of FIG. 3, for example, a virtual straight line (Not illustrated. The same applies to the following.) passing through the first point P1 and the second point P2, a virtual straight line passing through the point P3 and the point P4, a virtual line passing through the point P5 and the point P6, a virtual line passing through the point P7 and the point P8, and a virtual straight line passing through the point P9 and the point P10 each extends in the direction HO described above, namely, parallel to the direction HO.

As illustrated in FIGS. 3 and 5, the luminance signal generating unit KSS generates a first luminance signal KI1, a second luminance signal KI2, a third luminance signal KI3, and a fourth luminance signal KI4 using the luminance at the first point P1, the luminance at the second point P2, the weight function A, and a weight function B.

The weight function A and the weight function B correspond to the “first distribution” and the “second distribution”, respectively.

More specifically, the luminance signal generating unit KSS superimposes the luminance of the first point P1 in a measurement area R and the luminance of the second point P2 in the measurement area R on the weight function A. As a result, as illustrated in FIG. 3, the luminance signal generating unit KSS generates the first luminance signal KI1 indicating the luminance after being weighted by the weight function A at the first point P1 and the second luminance signal KI2 indicating the luminance after being weighted by the weight function A at the second point P2.

The luminance signal generating unit KSS also superimposes the luminance of the first point P1 in the measurement area R and the luminance of the second point P2 in the measurement area R on the weight function B instead of the weight function A. As a result, as illustrated in FIG. 3, the luminance signal generating unit KSS generates the third luminance signal KI3 indicating the luminance after being weighted by the weight function B at the first point P1 and the fourth luminance signal KI4 indicating the luminance after being weighted by the weight function B at the second point P2.

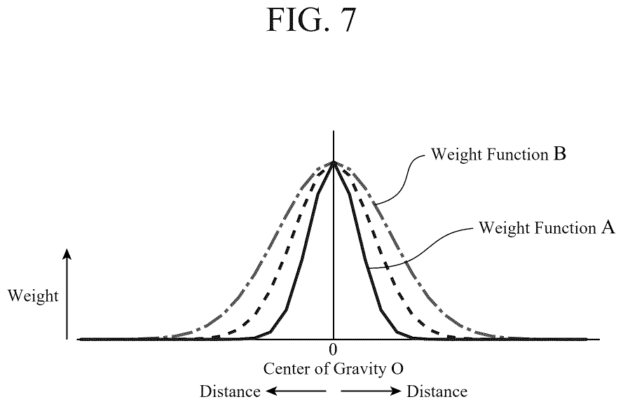

FIG. 7 is a graph illustrating the structures of the weight function A and the weight function B of the first embodiment.

In FIG. 7, the horizontal axis indicates the position on coordinates, namely, the distance from the center of gravity O, and the vertical axis indicates the magnitude of weighting. As illustrated in FIG. 7, the weight function A and the weight function B have spatially different weights. Specifically, the weight function A has a small spread of weight, which is the degree of spread of the weight (vertical axis) with respect to the distance (horizontal axis), whereas the weight function B has a wide spread of weight. More specifically, comparison between the weight function A and the weight function B shows that, in the weight function A, weighting values are concentrated within a range of a distance shorter than that of the weight function B, namely, in the weight function B, the weighting values are diffused within a range of a distance longer than that of the weight function A. In other words, in the weight function A, the weighting value is substantially 0 at a position of a relatively short distance, whereas in the weight function B, the weighting value is substantially 0 at a position of a relatively long distance.

For example, a two-dimensional Gaussian function can be used as the weight function A and the weight function B. The weight function A and the weight function B using the two-dimensional Gaussian function can be expressed by using, for example, the position of coordinates, a weight coefficient at the position of coordinates, a coordinate position of the center of gravity, and the manner how the Gaussian function spreads as parameters.

Referring back to FIG. 1, the description will be continued.

As illustrated in FIGS. 4 and 5, the pulse wave source signal generating unit MMS generates the first pulse wave source signal MM1 by combining the first luminance signal KI1 and the second luminance signal KI2. As illustrated in FIGS. 4 and 5, the pulse wave source signal generating unit MMS also generates the second pulse wave source signal MM2 by combining the third luminance signal KI3 and the fourth luminance signal KI4.

As illustrated in FIG. 5, the first pulse wave source signal MM1 has a characteristic that the peak of the pulse wave is emphasized, whereas the second pulse wave source signal MM2 has a characteristic that the peak of the pulse wave is not emphasized.

As illustrated in FIGS. 4 and 6, the pulse wave signal generating unit MSS generates the pulse wave signal MY indicating the pulse wave M of the subject HK by subtracting the second pulse wave source signal MM2 from the first pulse wave source signal MM1.

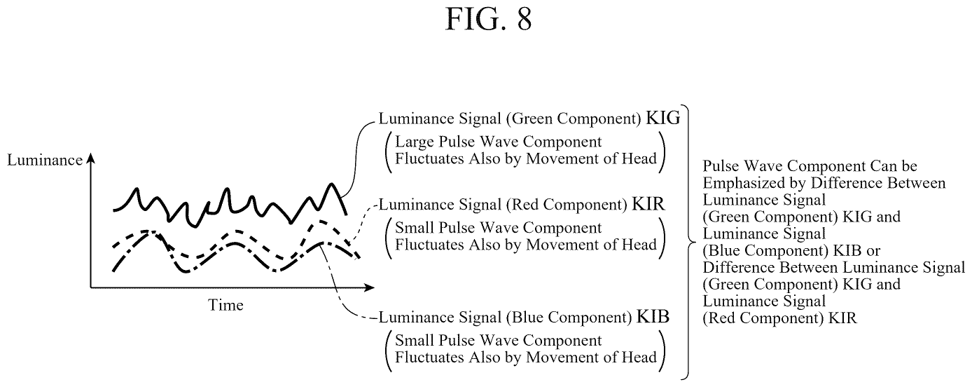

FIG. 8 is a graph illustrating luminance signals of a comparative example.

In the pulse wave detection device described in Patent Literature 1 as a comparative example, as illustrated in FIG. 8, a luminance signal (red component) KIR is subtracted from a luminance signal (green component) KIG, or a luminance signal (blue component) KIB is subtracted from the luminance signal (green component) KIG. The pulse wave detection device improves the accuracy of estimation of a pulse wave M of a subject HK by removing noise, which is an error in estimating the pulse wave M of the subject HK due to the movement of the head of the subject HK, by the subtraction.

The first pulse wave source signal MM1 described above corresponds to the luminance signal (green component) KIG of the comparative example, and the second pulse wave source signal MM2 corresponds to the luminance signal (red component) KIR or the luminance signal (blue component) KIB of the comparative example.

Referring back to FIG. 1, the description will be continued.

The pulse wave estimating unit MSU estimates the pulse wave M of the subject HK on the basis of the pulse wave signal MY (illustrated in FIG. 4 and FIG. 6). The pulse wave estimating unit MSU generates pulse wave information MJ indicating the pulse wave M.

The pulse wave information MJ indicates, for example, the pulse rate (the number of pulses per minute). The pulse wave estimating unit MSU uses, for example, a peak frequency in a frequency power spectrum obtained by performing Fourier transform on the pulse wave signal MY as the pulse rate that is the pulse wave information MJ.

### Configuration of First Embodiment

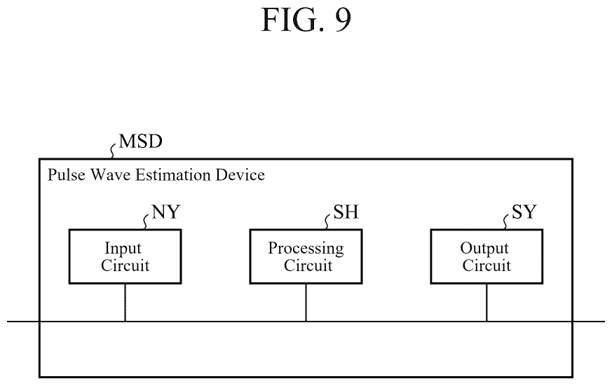

FIG. 9 is a diagram illustrating a hardware configuration of the pulse wave estimation device MSD of the first embodiment.

As illustrated in FIG. 9, the pulse wave estimation device MSD includes a processing circuit SH and further includes an input circuit NY and an output circuit SY as necessary.

The processing circuit SH is dedicated hardware. The processing circuit SH implements the functions of the image acquisition unit GSY to the pulse wave estimating unit MSU (illustrated in FIG. 1) of the pulse wave estimation device MSD.

The processing circuit SH includes, for example, a single circuit, a composite circuit, a programmed processor, a parallel programmed processor, an application specific integrated circuit (ASIC), a field-programmable gate array (FPGA), or a combination thereof.

The input circuit NY and the output circuit SY, for example, exchange input and output related to the operation of the processing circuit SH with the outside of the pulse wave estimation device MSD.

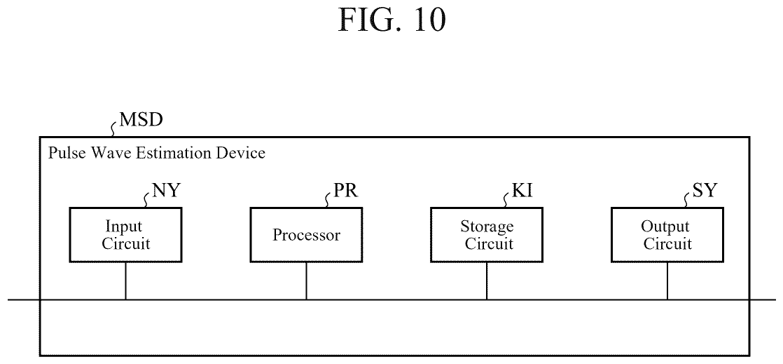

FIG. 10 is a diagram illustrating a hardware configuration based on implementation by software of the pulse wave estimation device MSD of the first embodiment.

As illustrated in FIG. 10, the pulse wave estimation device MSD includes a processor PR and a storage circuit KI and further includes an input circuit NY and an output circuit SY as necessary.

The processor PR is a central processing unit (also referred to as a CPU, a processing device, an arithmetic device, a microprocessor, a microcomputer, digital signal processing (DSP), or the like). The processor PR implements the functions of the image acquisition unit GSY to the pulse wave estimating unit MSU (illustrated in FIG. 1) of the pulse wave estimation device MSD.

The processor PR implements the above functions by software, firmware, or a combination of software and firmware. The software and the firmware are described as a program and stored in the storage circuit KI.

The processor PR implements the functions by reading and executing the program from the storage circuit KI. It can also be said that the program causes a computer to execute the procedures and methods of the image acquisition unit GSY to the pulse wave estimating unit MSU.

Here, the storage circuit KI is, for example, a nonvolatile or volatile semiconductor memory such as a random access memory (RAM), a read only memory (ROM), a flash memory, an erasable programmable read only memory (EPROM), or an electrically erasable programmable read only memory (EEPROM), a magnetic disc, a flexible disc, an optical disc, a compact disc, a mini disc, or a digital versatile disc (DVD).

Among the functions of the image acquisition unit GSY to the pulse wave estimating unit MSU (illustrated in FIG. 1), some functions may be implemented by the processing circuit SH (illustrated in FIG. 9), and other functions may be implemented by the processor PR (illustrated in FIG. 10).

As described above, the functions of the image acquisition unit GSY to the pulse wave estimating unit MSU of the pulse wave estimation device MSD can be implemented by hardware, software, firmware, or a combination thereof.

The input circuit NY and the output circuit SY, for example, exchange input and output related to the operation of the processor PR with the outside of the pulse wave estimation device MSD.

### Operation of First Embodiment

The operation of the pulse wave estimation device MSD of the first embodiment will be described.

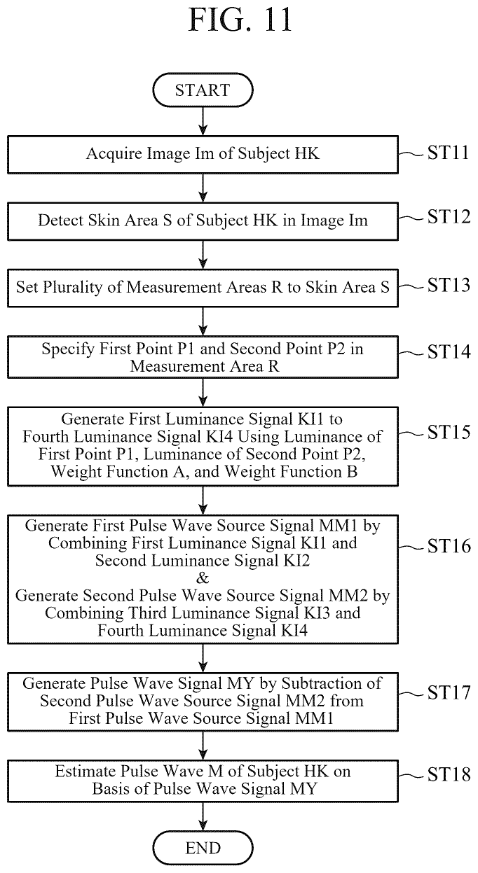

FIG. 11 is a flowchart illustrating the operation of the pulse wave estimation device MSD of the first embodiment. The operation of the pulse wave estimation device MSD of the first embodiment will be described by referring to the flowchart of FIG. 11.
Step ST11: The image acquisition unit GSY (illustrated in FIG. 1) acquires the image Im (illustrated in FIG. 1, FIG. 2A, and others) of the subject HK (illustrated in FIG. 1).
Step ST12: The skin area detecting unit HRK (illustrated in FIG. 1) detects the skin area S (illustrated in FIG. 1, FIG. 2B, and others) of the subject HK in the image Im of the subject HK. The skin area detecting unit HRK generates skin area information SJ indicating the skin area S of the subject HK.
Step ST13: The measurement area setting unit KRS (illustrated in FIG. 1) sets a plurality of measurement areas R (illustrated in FIG. 1, FIG. 2C, and others) in the skin area S of the subject HK.
Step ST14: The point specifying unit TTO (illustrated in FIG. 1) specifies the first point P1 and the second point P2 (illustrated in FIG. 3, FIG. 5, and others) in the measurement area R, for example, in the measurement area R56 (illustrated in FIG. 3) and in the direction HO (illustrated in FIG. 3) in which the pulse wave M of the subject HK is propagated.
Step ST15: The luminance signal generating unit KSS (illustrated in FIG. 1) generates the first luminance signal KI1 to the fourth luminance signal KI4 (illustrated in FIG. 3, FIG. 4, and others) using the luminance at the first point P1 in the measurement area R, the luminance at the second point P2 in the measurement area R (illustrated in FIG. 3), the weight function A, and the weight function B (illustrated in FIG. 3 and others).
Step ST16: The pulse wave signal generating unit MSS (illustrated in FIG. 1) generates the first pulse wave source signal MM1 (illustrated in FIG. 4 and FIG. 5) by combining the first luminance signal KI1 and the second luminance signal KI2. The pulse wave source signal generating unit MMS also generates the second pulse wave source signal MM2 (illustrated in FIG. 4 and FIG. 5) by combining the third luminance signal KI3 and the fourth luminance signal KI4.
Step ST17: The pulse wave signal generating unit MSS (illustrated in FIG. 1) generates the pulse wave signal MY (illustrated in FIG. 4 and FIG. 6) indicating the pulse wave M of the subject HK by performing subtraction between the first pulse wave source signal MM1 and the second pulse wave source signal MM2, more specifically, by subtracting the second pulse wave source signal MM2 from the first pulse wave source signal MM1.
Step ST18: The pulse wave estimating unit MSU (illustrated in FIG. 1) estimates the pulse wave M of the subject HK (illustrated in FIG. 1) on the basis of the pulse wave signal MY. The pulse wave estimating unit MSU generates pulse wave information MJ (illustrated in FIG. 1) indicating the pulse wave M.

### Effects of First Embodiment

As described above, in the pulse wave estimation device MSD according to the first embodiment, the luminance signal generating unit KSS generates the first luminance signal KI1 to the fourth luminance signal KI4 on the basis of the luminance at the first point P1 in the measurement area R, the luminance at the second point P2 in the measurement area R, the weight function A, and the weight function B, the pulse wave source signal generating unit MMS generates the first pulse wave source signal MM1 and the second pulse wave source signal MM2 using the first luminance signal KI1 to the fourth luminance signal KI4, the pulse wave signal generating unit MSS generates the pulse wave signal MY using the first pulse wave source signal MM1 and the second pulse wave source signal MM2, and the pulse wave estimating unit MSU estimates the pulse wave M on the basis of the pulse wave signal MY. As a result, it is possible to improve the accuracy of estimation of the pulse wave M of the subject HK even while using the image Im of the subject HK photographed under the near-infrared light.

### Modification of First Embodiment

Next, a modification of the first embodiment will be described.

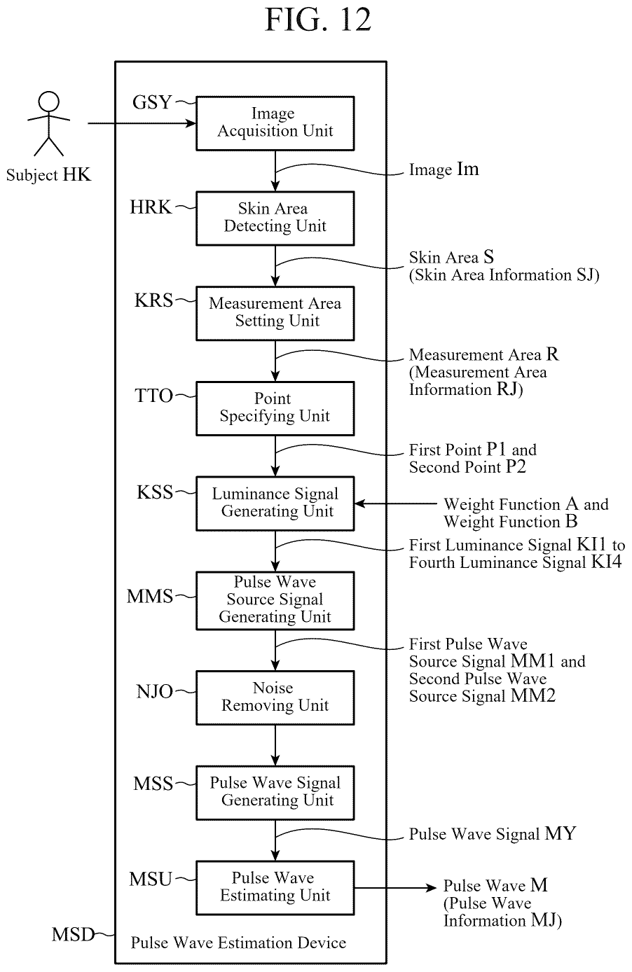

FIG. 12 is a diagram illustrating a configuration of a pulse wave estimation device MSD of a modification of the first embodiment.

The first pulse wave source signal MM1 and the second pulse wave source signal MM2 described in the first embodiment include various noise components NS (not illustrated) and include, for example, a noise component NS caused by a defect of some of elements included in the image acquisition unit GSY. In order to remove the noise component NS from the first pulse wave source signal MM1 and the second pulse wave source signal MM2, it is desirable to apply filter processing FS (not illustrated) to the first pulse wave source signal MM1 and the second pulse wave source signal MM2.

The pulse wave estimation device MSD according to the modification of the first embodiment further includes a noise removing unit NJO to perform the filter processing FS.

As illustrated in FIG. 12, the noise removing unit NJO is disposed between the pulse wave source signal generating unit MMS and the pulse wave signal generating unit MSS.

The noise removing unit NJO applies, for example, a low-pass filter, a high-pass filter, or a band-pass filter to the first pulse wave source signal MM1 and the second pulse wave source signal MM2 as the filter processing FS.

In the following description, it is based on the premise that the noise removing unit NJO applies a band pass filter as the filter processing FS.

The bandpass filter includes, for example, a Butterworth filter. In the band bus filter, it is desirable that the cutoff frequency on the low side is, for example, 0.5 Hz and that the cutoff frequency on the high side is, for example, 5.0 Hz.

The noise removing unit NJO removes the noise component NS included in the first pulse wave source signal MM1 and the second pulse wave source signal MM2 from the first pulse wave source signal MM1 and the second pulse wave source signal MM2 and outputs the first pulse wave source signal MM1 from which the noise component NS has been removed and the second pulse wave source signal MM2 from which the noise component NS has been removed to the pulse wave signal generating unit MSS.

In the pulse wave estimation device MSD according to the modification of the first embodiment, the noise removing unit NJO performs the filter processing FS on the first pulse wave source signal MM1 and the second pulse wave source signal MM2. As a result, the noise component NS included in the first pulse wave source signal MM1 and the second pulse wave source signal MM2 is removed. As a result, since the noise component NS is removed from the pulse wave signal MY generated by the pulse wave source signal generating unit MMS, the estimation accuracy of the pulse wave M of the subject HK by the pulse wave estimating unit MSU can be enhanced as compared to that in the first embodiment.

### Second Embodiment

### Second Embodiment

A pulse wave estimation device MSD according to a second embodiment will be described.

The pulse wave estimation device MSD of the second embodiment estimates a pulse wave M of a subject HK on the basis of two different measurement areas R, for example, measurement areas R1 and R2 (illustrated in FIG. 2C) unlike the pulse wave estimation device MSD (illustrated in FIG. 1) of the first embodiment that performs the estimation on the basis of the same measurement area R, for example, the measurement area R56 (illustrated in FIG. 6).

### Function of Second Embodiment

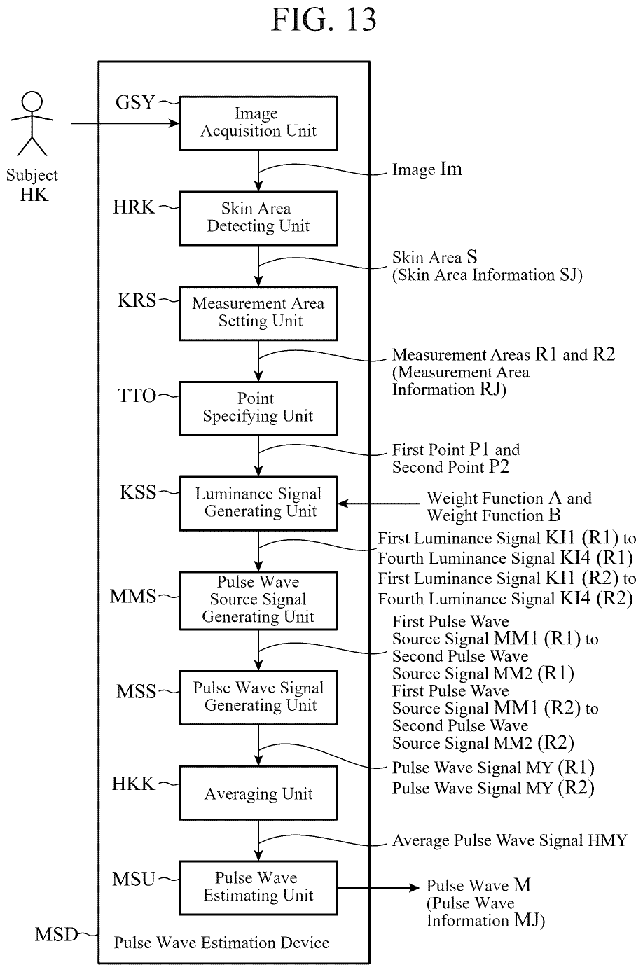

FIG. 13 is a functional block diagram of the pulse wave estimation device MSD of the second embodiment.

As is clear from the comparison between FIG. 13 and FIG. 1 (functional block diagram of the pulse wave estimation device MSD of the first embodiment), the pulse wave estimation device MSD of the second embodiment basically has a function similar to the function of the pulse wave estimation device MSD of the first embodiment. Meanwhile, the pulse wave estimation device MSD of the second embodiment further includes an averaging unit HKK, which is different from the pulse wave estimation device MSD of the first embodiment.

The averaging unit HKK corresponds to the “averaging unit”.

The averaging unit HKK averages a pulse wave signal MY (R1) for the measurement area R1 and a pulse wave signal MY (R2) for the measurement area R2.

### Configuration of Second Embodiment

The hardware configuration of the pulse wave estimation device MSD according to the second embodiment and the hardware configuration based on implementation by software are similar to the hardware configuration of the pulse wave estimation device MSD of the first embodiment (illustrated in FIG. 9) and the hardware configuration based on implementation by software (illustrated in FIG. 10).

### Operation of Second Embodiment

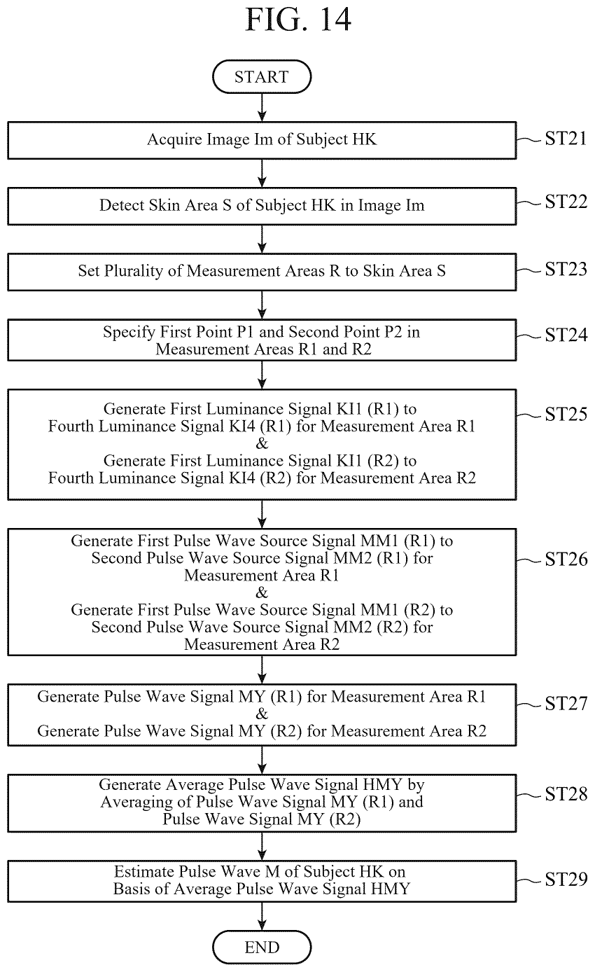

FIG. 14 is a flowchart illustrating the operation of the pulse wave estimation device MSD of the second embodiment.

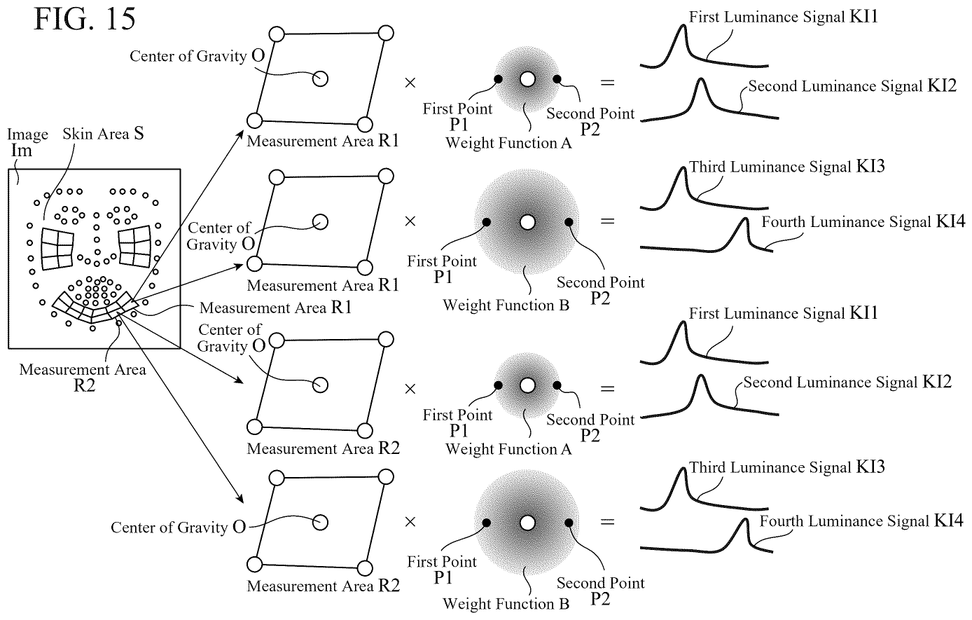

FIG. 15 is a diagram illustrating a first luminance signal KI1 (R1) to a fourth luminance signal KI4 (R1) of the pulse wave estimation device MSD of the second embodiment and operations up to generation of the first luminance signal KI1 (R2) to the fourth luminance signal KI4 (R2).

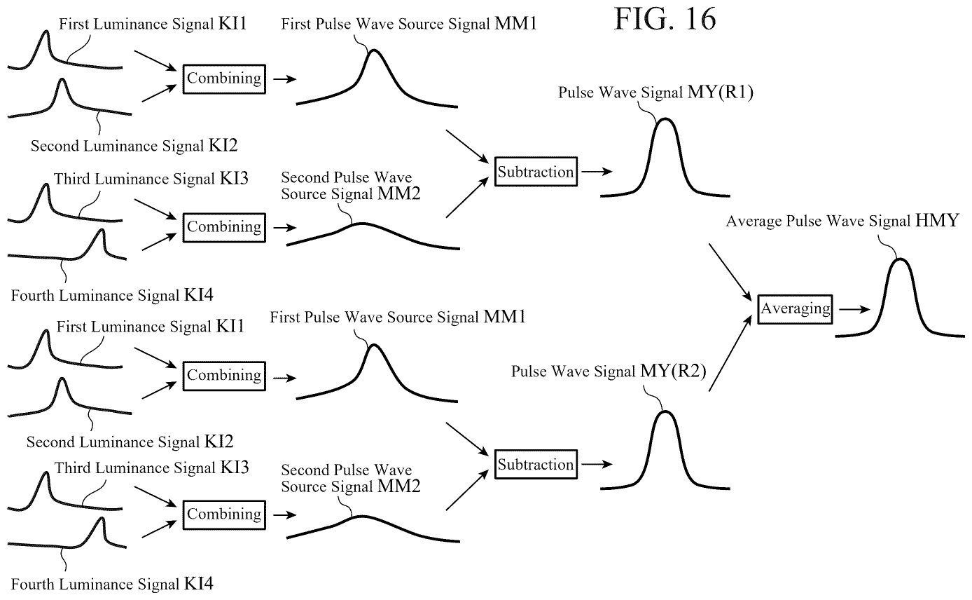

FIG. 16 is a diagram illustrating operations of the pulse wave estimation device MSD of the second embodiment up to generation of an average pulse wave signal HMY.

The operation of the pulse wave estimation device MSD of the second embodiment will be described by referring to FIGS. 14 to 16.
Step ST21: The image acquisition unit GSY (illustrated in FIG. 13) acquires an image Im (illustrated in FIG. 15) of the subject HK (illustrated in FIG. 13) similarly to step ST11 of the first embodiment.
Step ST22: The skin area detecting unit HRK (illustrated in FIG. 13) detects a skin area S (illustrated in FIG. 15) of the subject HK in the image Im of the subject HK similarly to step ST12 of the first embodiment. The skin area detecting unit HRK generates skin area information SJ indicating the skin area S of the subject HK.
Step ST23: The measurement area setting unit KRS (illustrated in FIG. 13) sets a plurality of measurement areas R (illustrated in FIG. 15) in the skin area S of the subject HK similarly to step ST13 of the first embodiment.
Step ST24: Unlike step ST14 of the first embodiment, as illustrated in FIG. 15, the point specifying unit TTO (illustrated in FIG. 13) specifies a first point P1 and a second point P2 in the measurement area R1 in a direction HO in which the pulse wave M of the subject HK is propagated (for example, illustrated in FIG. 3 of the first embodiment) and further specifies a first point P1 and a second point P2 in the measurement area R2, which is different from the measurement area R1, in the direction HO in which the pulse wave M of the subject HK is propagated.
Step ST25: Unlike step ST15 of the first embodiment, as illustrated in FIG. 15, the luminance signal generating unit KSS (illustrated in FIG. 13) generates the first luminance signal KI1 (R1) to the fourth luminance signal KI4 (R1) using the luminance at the first point P1 in the measurement area R1, the luminance at the second point P2 in the measurement area R1, a weight function A, and a weight function B and further generates the first luminance signal KI1 (R2) to the fourth luminance signal KI4 (R2) using the luminance at the first point P1 in the measurement area R2, the luminance at the second point P2 in the measurement area R2, the weight function A, and the weight function B.
Step ST26: Unlike step ST16 of the first embodiment, as illustrated in FIG. 16, the pulse wave signal generating unit MSS (illustrated in FIG. 13) generates a first pulse wave source signal MM1 (R1) by combining the first luminance signal KI1 (R1) and the second luminance signal KI2 (R1) for the measurement area R1 and generates a second pulse wave source signal MM2 (R1) by combining the third luminance signal KI3 (R1) and the fourth luminance signal KI4 (R1). Meanwhile, for the measurement area R2, the pulse wave signal generating unit MSS generates a first pulse wave source signal MM1 (R2) by combining the first luminance signal KI1 (R2) and the second luminance signal KI2 (R2) and generates a second pulse wave source signal MM2 (R2) by combining the third luminance signal KI3 (R2) and the fourth luminance signal KI4 (R2).
Step ST27: Unlike step ST17 of the first embodiment, as illustrated in FIG. 16, the pulse wave signal generating unit MSS (illustrated in FIG. 13) generates the pulse wave signal MY (R1) that can indicate the pulse wave M of the subject HK by subtracting the second pulse wave source signal MM2 (R1) from the first pulse wave source signal MM1 (R1) for the measurement area R1. Meanwhile, the pulse wave signal generating unit MSS generates the pulse wave signal MY (R2) that can indicate the pulse wave M of the subject HK by subtracting the second pulse wave source signal MM2 (R2) from the first pulse wave source signal MM1 (R2) for the measurement area R2.
Step ST28: As illustrated in FIG. 16, the averaging unit HKK (illustrated in FIG. 13) averages the pulse wave signal MY (R1) for the measurement area R1 and the pulse wave signal MY (R2) for the measurement area R2. As a result, as illustrated in FIG. 16, the averaging unit HKK generates the average pulse wave signal HMY that is one averaged signal.
Step ST29: The pulse wave estimating unit MSU (illustrated in FIG. 13) estimates the pulse wave M of the subject HK on the basis of the average pulse wave signal HMY. The pulse wave estimating unit MSU generates pulse wave information MJ indicating the pulse wave M.

### Effects of Second Embodiment

As described above, in the pulse wave estimation device MSD of the second embodiment, the image acquisition unit GSY to the pulse wave signal generating unit MSS cooperatively generate the pulse wave signal MY (R1) and the pulse wave signal MY (R2) for two mutually different measurement areas R, namely, the measurement areas R1 and R2, and the averaging unit HKK generates the average pulse wave signal HMY, which is one averaged signal, by averaging the pulse wave signal MY (R1) and the pulse wave signal MY (R2). The pulse wave estimating unit MSU estimates the pulse wave M of the subject HK on the basis of the average pulse wave signal HMY instead of the pulse wave signal MY of the first embodiment. As a result, for example, even in a case where the measurement area R1 is partially blocked due to the presence of an obstacle between the subject HK and the pulse wave estimation device MSD, namely, even in a case where a part of the measurement area R1 is not captured in the image Im of the subject HK, the pulse wave M of the subject HK can be estimated with a similar accuracy to that in the first embodiment by using the measurement area R2 in combination.

### Third Embodiment

### Third Embodiment

A pulse wave estimation device MSD according to a third embodiment will be described.

The pulse wave estimation device MSD of the third embodiment subtracts the product of multiplication of a second pulse wave source signal MM2 by a coefficient (multiplication coefficient c described later) from a first pulse wave source signal MM1 in order to estimate a pulse wave M of a subject HK unlike the pulse wave estimation device MSD of the first embodiment that subtracts the second pulse wave source signal MM2 from the first pulse wave source signal MM1.

### Function of Third Embodiment

The pulse wave estimation device MSD of the third embodiment basically has functions (illustrated in FIG. 1) similar to those of the pulse wave estimation device MSD of the first embodiment. Meanwhile, in the pulse wave estimation device MSD of the third embodiment, unlikely the pulse wave estimation device MSD of the first embodiment, a pulse wave source signal generating unit MMS of the third embodiment has a function different from that of the pulse wave source signal generating unit MMS of the first embodiment.

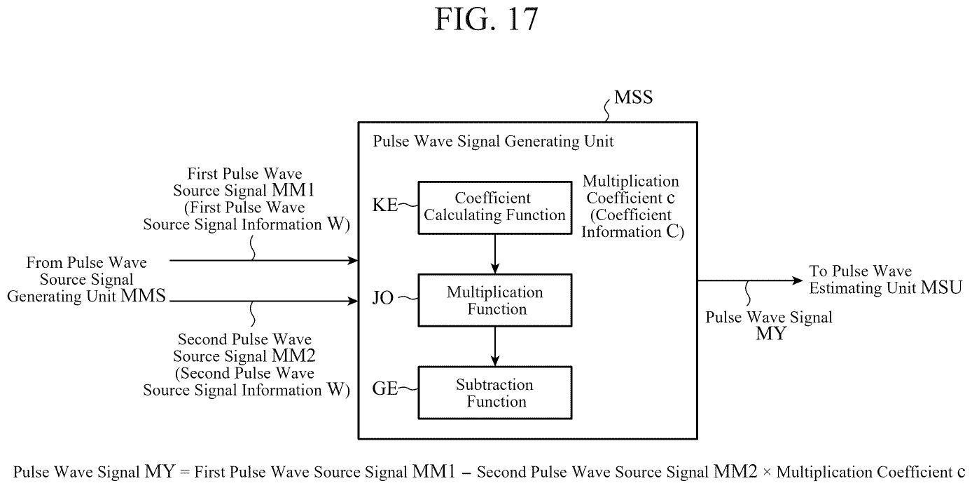

FIG. 17 is a functional block diagram of the pulse wave source signal generating unit MMS of the third embodiment.

As illustrated in FIG. 17, the pulse wave source signal generating unit MMS of the third embodiment includes a coefficient calculating function KE, a multiplication function JO, and a subtraction function GE.

The pulse wave source signal generating unit MMS of the third embodiment also handles first pulse wave source signal information W, second pulse wave source signal information M, and coefficient information C.

A relationship between each piece of information and each signal is as follows.
(A) The first pulse wave source signal information W indicates the first pulse wave source signal MM1.
(B) The second pulse wave source signal information M indicates the second pulse wave source signal MM2.
(C) The coefficient information C indicates the multiplication coefficient c.

The multiplication coefficient c corresponds to the “coefficient”.

The pulse wave signal generating unit MSS generates a pulse wave signal MY on the basis of the first pulse wave source signal information W and the second pulse wave source signal information M, in other words, generates the pulse wave signal MY on the basis of the first pulse wave source signal MM1 and the second pulse wave source signal MM2.

More specifically, the pulse wave signal generating unit MSS performs the following.
(1) As the coefficient calculating function KE, calculates the coefficient information C indicating the multiplication coefficient c to be multiplied to the second pulse wave source signal information M on the basis of the first pulse wave source signal information W and the second pulse wave source signal information M.
(2) As the multiplication function JO, multiply the second pulse wave source signal MM2 by the multiplication coefficient c described in (1).
(3) Subtract the product of the multiplication of (2) from the first pulse wave source signal MM1.

The pulse wave signal generating unit MSS generates the pulse wave signal MY by performing (1) to (3).

The coefficient information C indicates the amount, namely, the magnitude of the second pulse wave source signal information M to be subtracted.

More specifically, the value of the multiplication coefficient c is adjusted in such a manner that a component necessary for estimating the pulse wave M of the subject HK remains in the pulse wave signal MY obtained by subtracting the product of the multiplication of the second pulse wave source signal MM2 by the multiplication coefficient c from the first pulse wave source signal MM1.

In a case where the value of the multiplication coefficient c is large, the product of the multiplication of the second pulse wave source signal MM2 by the multiplication coefficient c is large. In contrast, in a case where the multiplication coefficient c is small, the product of the multiplication of the second pulse wave source signal MM2 by the multiplication coefficient c is small.

The pulse wave signal generating unit MSS calculates the coefficient information C in such a manner as to reduce the difference between the first pulse wave source signal information W and a product of multiplication of the second pulse wave source signal information M by the coefficient information C. As a result, the pulse wave signal generating unit MSS can adjust the amount of the second pulse wave source signal information M contained in the first pulse wave source signal information W by the coefficient information C.

### Configuration of Third Embodiment

The hardware configuration of the pulse wave estimation device MSD according to the third embodiment and the hardware configuration based on implementation by software are similar to the hardware configuration of the pulse wave estimation device MSD of the first embodiment (illustrated in FIG. 9) and the hardware configuration based on implementation by software (illustrated in FIG. 10).

### Operation of Third Embodiment

The operation of the pulse wave estimation device MSD of the third embodiment is basically similar to the operation of the pulse wave estimation device MSD of the first embodiment (illustrated in FIG. 11). Meanwhile, the pulse wave estimation device MSD of the third embodiment is different from the pulse wave estimation device MSD of the first embodiment in the operation of the pulse wave signal generating unit MSS. Hereinafter, the operation of the pulse wave signal generating unit MSS of the third embodiment will be mainly described.

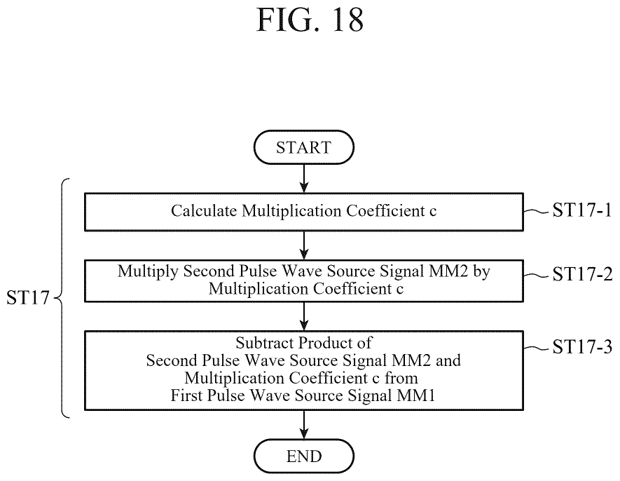

FIG. 18 is a flowchart illustrating the operation of the pulse wave estimation device MSD of the third embodiment. The operation of the pulse wave estimation device MSD of the third embodiment will be described by referring to the flowchart of FIG. 18.
Step ST17-1: The pulse wave signal generating unit MSS (illustrated in FIG. 17) receives the first pulse wave source signal MM1 and the second pulse wave source signal MM2 (illustrated in FIG. 17, FIG. 1, and FIG. 4) from the pulse wave source signal generating unit MMS (illustrated in FIG. 1) as a preceding stage. The pulse wave signal generating unit MSS calculates, as the coefficient calculating function KE, the multiplication coefficient c that is a coefficient on the basis of the first pulse wave source signal information W and the second pulse wave source signal Information M.
Step ST17-2: The pulse wave signal generating unit MSS multiplies, as the multiplication function JO, the second pulse wave source signal MM2 by the multiplication coefficient c calculated in step ST17-1 described above.
Step ST17-3: The pulse wave signal generating unit MSS subtracts, as the subtraction function GE, the product of the multiplication in step ST17-2 from the first pulse wave source signal MM1. The pulse wave signal generating unit MSS calculates the pulse wave signal MY by the subtraction. The pulse wave signal generating unit MSS outputs the calculated pulse wave signal MY to the pulse wave estimating unit MSU as a subsequent stage.
Step ST17-3 and subsequent steps: The pulse wave estimating unit MSU estimates the pulse wave M of the subject HK on the basis of the pulse wave signal MY received from the pulse wave signal generating unit MSS and generated through steps ST17-1 to ST17-3 described above.

### Effects of Third Embodiment

As described above, in the pulse wave estimation device MSD according to the third embodiment, the pulse wave signal generating unit MSS calculates the pulse wave signal MY by subtracting the product obtained by multiplying the second pulse wave source signal MM2 by the multiplication coefficient c from the first pulse wave source signal MM1. This makes it possible to avoid signal components useful for estimation of the pulse wave M of the subject HK included in the first pulse wave source signal MM1 and the second pulse wave source signal MM2 from being unnecessarily reduced. As a result, the pulse wave estimation device MSD of the third embodiment can improve the accuracy of estimating the pulse wave M of the subject HK as compared with the pulse wave estimation device MSD of the first embodiment which simply subtract the second pulse wave source signal MM2 from the first pulse wave source signal MM1.

### Fourth Embodiment

### Fourth Embodiment

A pulse wave estimation device MSD according to a fourth embodiment will be described.

The pulse wave estimation device MSD of the fourth embodiment does not use the weight functions in order to generate the first luminance signal KI1 to the fourth luminance signal KI4 unlike the pulse wave estimation device MSD (illustrated in FIG. 1) of the first embodiment that uses the weight functions such as the weight function A and the weight function B (for example, illustrated in FIG. 3).

### Function of Fourth Embodiment

The pulse wave estimation device MSD of the fourth embodiment basically has functions similar to those of the pulse wave estimation device MSD of the first embodiment. Meanwhile, the pulse wave estimation device MSD of the fourth embodiment is different from the pulse wave estimation device MSD of the first embodiment in that a measurement area setting unit KRS, a point specifying unit TTO, and a luminance signal generating unit KSS of the fourth embodiment are different from the measurement area setting unit KRS, the point specifying unit TTO, and the luminance signal generating unit KSS of the first embodiment in terms of the function.

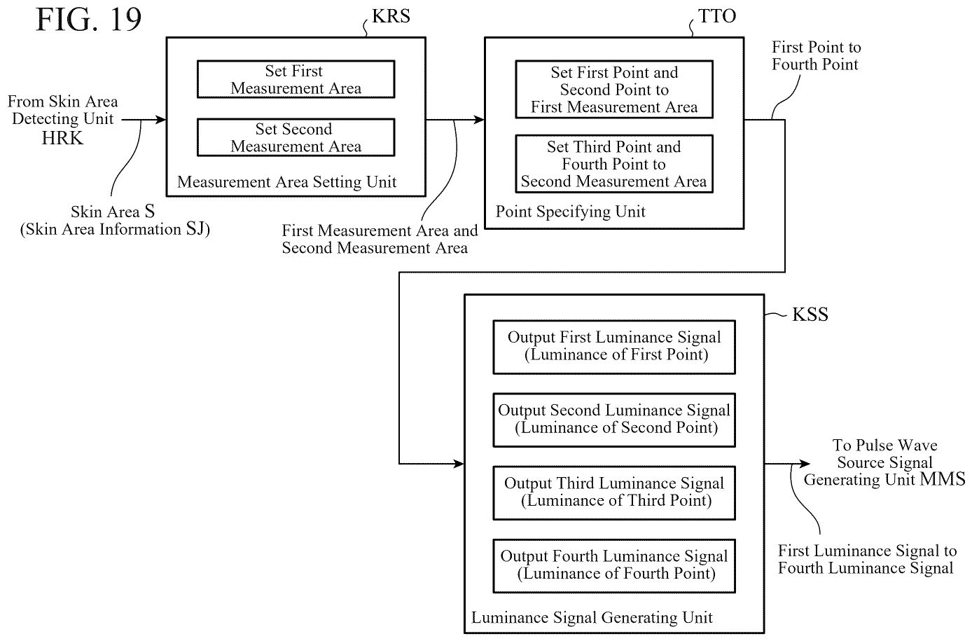

FIG. 19 is a functional block diagram of the measurement area setting unit KRS, the point specifying unit TTO, and the luminance signal generating unit KSS of the fourth embodiment.

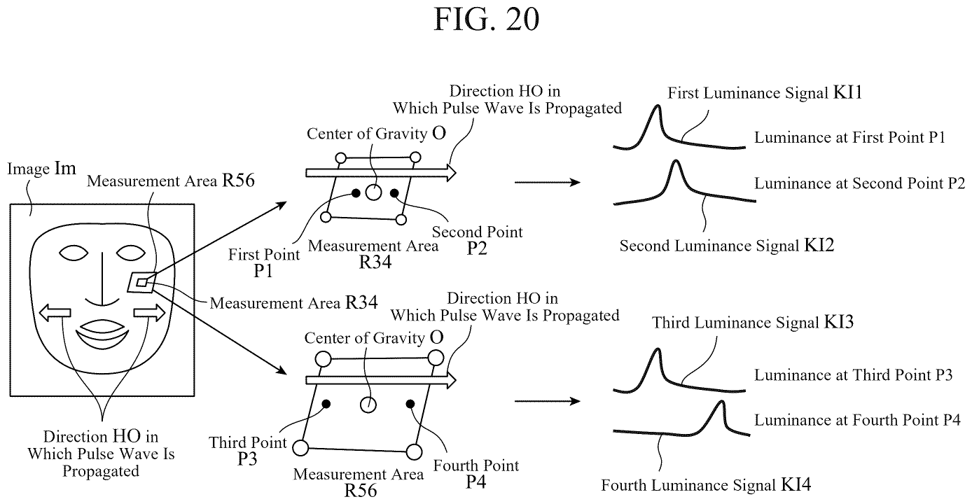

FIG. 20 is a diagram illustrating a measurement area R, a first point P1 to a fourth point P4, and a first luminance signal KI1 to a fourth luminance signal KI4 according to the fourth embodiment.

As illustrated in FIGS. 19 and 20, the measurement area setting unit KRS has a function of setting a first measurement area (for example, a measurement area R34) and a function of setting a second measurement area (for example, a measurement area R56).

Here, as illustrated in FIG. 20, in the measurement area R34 and the measurement area R56, the center of gravity O, which is a reference position on the coordinates of the measurement area R34, and the center of gravity O, which is a reference position on the coordinates of the measurement area R56, are common to each other. The measurement area R34 and the measurement area R56 have a relationship in which the measurement area R56 is wider than the measurement area R34. Instead of the above relationship, the measurement area R56 may be narrower than the measurement area R34. The measurement area R34 and the measurement area R56 share the correspondence relationship between the position on the coordinates and the magnitude of weighting specified by the position on the coordinates and share, for example, one weight function (for example, the weight function A or the weight function B (illustrated in FIG. 7)).

As illustrated in FIG. 20, the point specifying unit TTO specifies a first point P1 and a second point P2 in the first measurement area, for example, in the measurement area R34, in a direction HO in which a pulse wave M of a subject HK is propagated. As illustrated in FIG. 20, the point specifying unit TTO sets a third point P3 and a fourth point P4 in the second measurement area, for example, in the measurement area R56 in the direction HO in which the pulse wave M of the subject HK is propagated.

As illustrated in FIG. 20, since the measurement area R56 is wider than the measurement area R34, the distance between the third point P3 and the fourth point P4 is longer than the distance between the first point P1 and the second point P2. In contrast to the above, in a case where the measurement area R56 is narrower than the measurement area R34, the distance between the third point P3 and the fourth point P4 is shorter than the distance between the first point P1 and the second point P2.

As illustrated in FIG. 20, the luminance signal generating unit KSS outputs the first luminance signal KI1 indicating the luminance of the first point P1 in the first measurement area, for example, the measurement area R34, and the second luminance signal KI2 indicating the luminance of the second point P2 in the measurement area R34. As illustrated in FIG. 20, the pulse wave signal generating unit MSS outputs the third luminance signal KI3 indicating the luminance of the third point P3 in the second measurement area, for example, the measurement area R56, and the fourth luminance signal KI4 indicating the luminance of the fourth point P4 in the measurement area R56.

### Configuration of Fourth Embodiment

The hardware configuration of the pulse wave estimation device MSD according to the fourth embodiment and the hardware configuration based on implementation by software are similar to the hardware configuration of the pulse wave estimation device MSD of the first embodiment (illustrated in FIG. 9) and the hardware configuration based on implementation by software (illustrated in FIG. 10).

### Operation of Fourth Embodiment

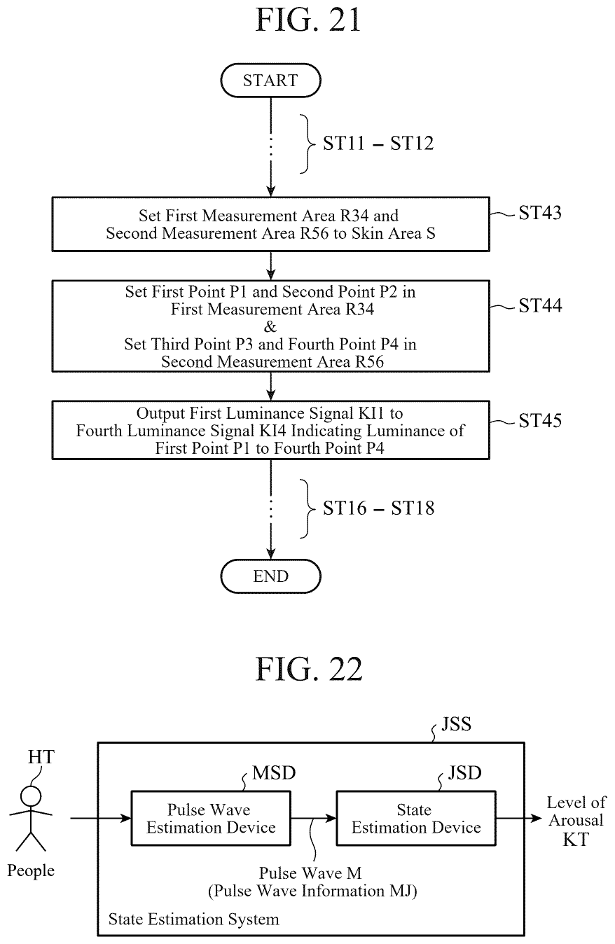

FIG. 21 is a flowchart illustrating the operation of the pulse wave estimation device MSD of the fourth embodiment. The operation of the pulse wave estimation device MSD of the fourth embodiment will be described by referring to the flowchart of FIG. 21.
Step ST43 and preceding steps: The pulse wave estimation device MSD of the fourth embodiment performs steps ST11 and ST12 (illustrated in FIG. 11).
Step ST43: As illustrated in FIG. 20, the measurement area setting unit KRS (illustrated in FIG. 19) sets the first measurement area, namely, the measurement area R34 in the skin area S. As illustrated in FIG. 20, the measurement area setting unit KRS sets the second measurement area, namely, the measurement area R56 in the skin area S.
Step ST44: As illustrated in FIG. 20, the point specifying unit TTO (illustrated in FIG. 19) sets the first point P1 and the second point P2 in the first measurement area, namely, the measurement area R34. As illustrated in FIG. 20, the point specifying unit TTO also sets the first point P1 and second point P2 in the second measurement area, namely, the measurement area R56.
Step ST45: As illustrated in FIG. 20, the luminance signal generating unit KSS (illustrated in FIG. 19) outputs the first luminance signal KI1 indicating the luminance of the first point P in the first measurement area, namely, in the measurement area R34, and the second luminance signal KI2 indicating the luminance of the second point P in the measurement area R34. As illustrated in FIG. 20, the luminance signal generating unit KSS also outputs the third luminance signal KI3 indicating the luminance of the third point P3 in the second measurement area, namely, the measurement area R56, and the fourth luminance signal KI4 indicating the luminance of the fourth point P4 in the measurement area R56.
Step ST45 and subsequent steps: The pulse wave estimation device MSD of the fourth embodiment performs steps ST16 to ST18 (illustrated in FIG. 11).

### Effects of Fourth Embodiment

As described above, in the pulse wave estimation device MSD of the fourth embodiment, the first luminance signal KI1 to the fourth luminance signal KI4 are generated similarly to the pulse wave estimation device MSD of the first embodiment without using the weight function A nor the weight function B of the pulse wave estimation device MSD of the first embodiment. As a result, the pulse wave estimation device MSD according to the fourth embodiment can improve the accuracy of estimating the pulse wave M of the subject HK even while using the image Im of the subject HK captured under near-infrared light, similarly to the pulse wave estimation device MSD of the first embodiment.

### Fifth Embodiment

### Fifth Embodiment

A state estimation system JSS according to a fifth embodiment will be described.

### Function of Fifth Embodiment

FIG. 22 is a functional block diagram of a state estimation system JSS of the fifth embodiment.

As illustrated in FIG. 22, the state estimation system JSS according to the fifth embodiment includes a pulse wave estimation device MSD and a state estimation device JSD.

The pulse wave estimation device MSD is one of the pulse wave estimation devices MSD of the first embodiment to the fourth embodiment described above. The pulse wave estimation device MSD outputs a pulse wave M estimated for a person HT (for example, a driver of a vehicle), more accurately, pulse wave information MJ of the person HT (for example, a pulse rate of the person HT), similarly to the pulse wave estimation devices MSD of the first to fourth embodiments outputting the pulse wave M estimated for the subject HK (for example, illustrated in FIG. 1).

The state estimation device JSD estimates the state of the person HT on the basis of the pulse wave information MJ of the person HT, and more specifically, for example, estimates the level of arousal KT of the driver of the vehicle. The level of arousal KT is represented by, for example, two-stage states (1: Drowsy state and 2: Awake state).

A driver of a vehicle is typically in the awake state immediately after starting driving of the vehicle. Therefore, the state estimation device JSD estimates which of the two-stage states the driver of the vehicle is in by referring to, for example, the pulse rate from a time point at which the driving of the vehicle has been started to a time point at which 10 minutes has elapsed as a reference. More specifically, the state estimation device JSD estimates that the level of arousal KT of the driver of the vehicle is “1: drowsy state” in a case where it is recognized that the pulse rate of the driver of the vehicle is extremely lower than the reference.

The state estimation device JSD may issue, for example, a warning sound together with the level of arousal KT that is “1: drowsy state” described above.

The person HT may be, for example, a person in a house (for example, a person sitting in a living room) or a person working in an office (for example, a person who is riding in an elevator) other than the driver of the vehicle.

### Effects of Fifth Embodiment

As described above, the state estimation system JSS according to the fifth embodiment can estimate the state of the person HT by including one of the pulse wave estimation devices MSD according to the first to fourth embodiments.

The above-described embodiments may be combined without departing from the gist of the present disclosure, and components in each of the embodiments may be deleted, modified, or added with another component as appropriate.

### INDUSTRIAL APPLICABILITY

The pulse wave estimation device and others of the present disclosure can be used to improve accuracy of estimating a pulse wave of a subject even while using an image captured under near-infrared light.

### REFERENCE SIGNS LIST

A: weight function, B: weight function, GSY: image acquisition unit, HK: subject, HRK: skin area detecting unit, Im: image, KI1: first luminance signal, KI2: second luminance signal, KI3: third luminance signal, KI4: fourth luminance signal, KRS: measurement area setting unit, KSS: luminance signal generating unit, M: pulse wave, MM1: first pulse wave source signal, MM2: second pulse wave source signal, MMS: pulse wave source signal generating unit, MSD: pulse wave estimation device, MSS: pulse wave signal generating unit, MSU: pulse wave estimating unit, MY: pulse wave signal, P1: first point, P2: second point, R: measurement area, S: skin area, TTO: point specifying unit

1. A pulse wave estimation device comprising:
processing circuitry configured to
acquire an image in which a person is photographed under near-infrared light;
detect a skin area from the image, the skin area being a position of skin of the person;
set a plurality of measurement areas in the skin area, the plurality of measurement areas being areas for measuring luminance of the skin of the person;
specify a first point and a second point in one of the plurality of measurement areas in a direction in which a pulse wave of the person is propagated in the image;
for a first distribution and a second distribution having mutually different correspondence relationships between a position on coordinates and a magnitude of weighting specified by the position on the coordinates,
generate a first luminance signal indicating luminance weighted by the first distribution at the first point and a second luminance signal indicating luminance weighted by the first distribution at the second point by superimposing the luminance of the first point and the luminance of the second point on the first distribution; and
generate a third luminance signal indicating luminance weighted by the second distribution at the first point and a fourth luminance signal indicating luminance weighted by the second distribution at the second point by superimposing the luminance of the first point and the luminance of the second point on the second distribution;
generate a first pulse wave source signal by combining the first luminance signal and the second luminance signal; and
generate a second pulse wave source signal by combining the third luminance signal and the fourth luminance signal;
generate a pulse wave signal indicating the pulse wave of the person by subtracting one of the first pulse wave source signal and the second pulse wave source signal from the other; and
estimate the pulse wave of the person on a basis of the pulse wave signal.

2. The pulse wave estimation device according to claim 1,
wherein the processing circuitry is further configured to
generate one averaged pulse wave signal by averaging a plurality of pulse wave signals generated in the plurality of measurement areas; and
estimate the pulse wave of the person on a basis of the one averaged pulse wave signal.

3. The pulse wave estimation device according to claim 1,
wherein the processing circuitry generates the pulse wave signal by:
calculating a coefficient for adjusting the second pulse wave source signal in such a manner that a signal component for estimating the pulse wave of the person remains in the pulse wave signal after subtracting the second pulse wave source signal from the first pulse wave source signal;
multiplying the second pulse wave source signal by the coefficient; and
subtracting a product of the multiplication from the first pulse wave source signal.

4. The pulse wave estimation device according to claim 1,
wherein the processing circuitry is further configured to set, as the plurality of measurement areas, the one measurement area and another measurement area having a common reference position on coordinates with the one measurement area, the other measurement area being wider or narrower than the one measurement area, the other measurement area allowing the one measurement area and the other measurement area to share a correspondence relationship between a position on the coordinates and a magnitude of weighting specified by the position on coordinates;
specify a third point and a fourth point in the other measurement area in a direction in which the pulse wave of the person is propagated, a distance between the third point and the fourth point being longer or shorter than a distance between the first point and the second point; and
output a first luminance signal indicating luminance of the first point in the one measurement area, a second luminance signal indicating luminance of the second point in the one measurement area, a third luminance signal indicating luminance of the third point in the other measurement area, and a fourth luminance signal indicating luminance of the fourth point in the other measurement area, instead of generating the first luminance signal, the second luminance signal, the third luminance signal, and the fourth luminance signal using the first distribution and the second distribution.

5. A state estimation system comprising:
the pulse wave estimation device according to claim 1; and
a state estimation device to estimate a state of the person on a basis of a pulse wave output from the pulse wave estimation device.

6. A pulse wave estimation method comprising:
acquiring an image in which a person is photographed under near-infrared light;
detecting a skin area from the image, the skin area being a position of skin of the person;
setting a plurality of measurement areas in the skin area, the plurality of measurement areas being areas for measuring luminance of the skin of the person;
specifying a first point and a second point in one of the plurality of measurement areas in a direction in which a pulse wave of the person is propagated in the image;
for a first distribution and a second distribution having mutually different correspondence relationships between a position on coordinates and a magnitude of weighting specified by the position on the coordinates,
generating a first luminance signal indicating luminance weighted by the first distribution at the first point and a second luminance signal indicating luminance weighted by the first distribution at the second point by superimposing the luminance of the first point and the luminance of the second point on the first distribution; and
generating a third luminance signal indicating luminance weighted by the second distribution at the first point and a fourth luminance signal indicating luminance weighted by the second distribution at the second point by superimposing the luminance of the first point and the luminance of the second point on the second distribution;
generating a first pulse wave source signal by combining the first luminance signal and the second luminance signal; and
generating a second pulse wave source signal by combining the third luminance signal and the fourth luminance signal;
generating a pulse wave signal indicating the pulse wave of the person by subtracting one of the first pulse wave source signal and the second pulse wave source signal from the other; and
estimating the pulse wave of the person on a basis of the pulse wave signal.

7. The pulse wave estimation method according to claim 6, further comprising
generating one averaged pulse wave signal by averaging a plurality of pulse wave signals generated in the plurality of measurement areas and
estimating the pulse wave of the person on a basis of the one averaged pulse wave signal.

8. The pulse wave estimation method according to claim 6,
wherein the pulse wave signal is generated by:
calculating a coefficient for adjusting the second pulse wave source signal in such a manner that a signal component for estimating the pulse wave of the person remains in the pulse wave signal after subtracting the second pulse wave source signal from the first pulse wave source signal;
multiplying the second pulse wave source signal by the coefficient; and
subtracting a product of the multiplication from the first pulse wave source signal.

9. The pulse wave estimation method according to claim 6, the method further comprising:
setting, as the plurality of measurement areas, the one measurement area and another measurement area having a common reference position on coordinates with the one measurement area, the other measurement area being wider or narrower than the one measurement area, the other measurement area allowing the one measurement area and the other measurement area to share a correspondence relationship between a position on the coordinates and a magnitude of weighting specified by the position on coordinates,
specifying a third point and a fourth point in the other measurement area in a direction in which the pulse wave of the person is propagated, a distance between the third point and the fourth point being longer or shorter than a distance between the first point and the second point; and
outputting a first luminance signal indicating luminance of the first point in the one measurement area, a second luminance signal indicating luminance of the second point in the one measurement area, a third luminance signal indicating luminance of the third point in the other measurement area, and a fourth luminance signal indicating luminance of the fourth point in the other measurement area, instead of generating the first luminance signal, the second luminance signal, the third luminance signal, and the fourth luminance signal using the first distribution and the second distribution.

10. A state estimation method comprising:
the pulse wave estimation method according to claim 6; and
a state estimation method of estimating a state of the person on a basis of the pulse wave output by the pulse wave estimation method.
